(19) United States
(12) Patent Application Publication
(10) Pub. No.: US 2008/0286344 A1
(43) Pub. Date: Nov. 20, 2008

(54) Solid form

(76) Inventors: Olivia Darmuzey, Brussels (BE); Graeme MacLeod, Wezembeek Oppem (BE); Dzenana Cengic, Brussels (BE); Kevin M. Stokes, Hamilton Square, NJ (US)

Correspondence Address: PATENT ADMINISTRATOR;FMC CORPORATION, 1735 MARKET STREET, PHILADELPHIA, PA, 19103, US

(21) Appl. No.: 11/804,005

(22) Filed: May 16, 2007

(51) Int. Cl. A61K 9/70; A61K 9/14; A61K 9/22; A61P 11/00; A61P 29/00; A61P 31/00; A61P 9/00; A61P 33/00; A61P 3/00; A61P 25/00; A61P 1/00; A61K 9/24

(52) U.S. Cl. 424/443; 424/468; 424/472; 424/489; 426/282; 510/441

(57) ABSTRACT

A solid form comprising at least one film enrobing a compacted fill material wherein:
i) the compacted fill material comprises at least one active material;
ii) the solid form shows a weight loss that is less than 1% during a 30 minutes USP friability test United States Pharmacopeia (USP) 29 Test Number 1216 (page 3046);
iii) the compacted fill material has a density of at least 0.5 g/ml based on the total solid volume of the solid form and a tensile strength of less than 0.9 MPa; and
iv) the compacted fill material is present in the solid form in at least a first zone and a second zone and the active material is present in at least one of the zones.

### FIELD OF THE INVENTION

This invention relates to a solid form comprising a film enrobing a compacted fill material, which comprises a plurality of components disposed in discrete zones in the solid form and a method of producing the solid form.

### BACKGROUND TO THE INVENTION

Active ingredients, for example pharmaceutical, agrochemical and detergent active ingredients may be delivered through a wide range of solid forms including tablets and capsules. Conventional tablets generally are highly compacted and have relatively high densities. In conventional tablets, the active ingredient is generally compacted with other components in a blend to provide the requisite structural integrity for the tablet. Delivery of the active ingredient in use may however be unsatisfactory due to the compaction level and it is known to add excipients to the formulation to aid disintegration or dissolution of the tablet to improve delivery, aid compaction, increase strength and increase robustness of the solid form. This may however impose constraints on the flexibility of the formulator in developing tablets containing the active ingredient.

Capsules generally include the active ingredient in a relatively non-compacted form. However, the lack of compaction together with the void space inherent within capsules mean that for a given large dose of active, the volume of the final solid form is greater than for more compacted solid forms. Increasing the size of the capsule to accommodate the required dose is undesirable for the user. Typically, capsules require a relatively high level of disintegrant to provide adequate disintegration of the solid form. Capsule shells may also be sensitive to moisture and present problems as regards storage and product shelf-life.

WO 03/096963 discloses solid forms and processes utilizing films to enrobe a fill material to a degree of compaction less than that generally used to make a tablet. It is specifically disclosed therein that because of the nature of the capsule produced that certain ancillary ingredients necessary in conventional tablet production may be omitted. It is further disclosed therein that, due to relatively loose compaction, components contained within a tablet which are “designed to disperse and breakup the tablet when it has reached the site of delivery, can be omitted, as the active ingredients in the capsule according to the present invention are in a non-compacted or at least less compacted form as compared to a conventional tablet, and this lesser compaction leads to the easy release and dispersal of active ingredients once the capsule film has dissolved, e.g., at the intended site of delivery”.

Products comprising a plurality of components which are located in separate zones in the product formulation are known, for example, GB-A-1099999 discloses a two-layered multi-vitamin tablet having a vitamin in each layer. Separating components in a formulation may be desirable for a number of reasons, for example to provide sequential or controlled-release of the components and to avoid mutual incompatibility of the components.

WO0/38650 discloses a gastric retention solid form which is multi-layered and adapted for retention in the stomach. The solid form comprises a first layer of a swellable water-soluble polymer, a second layer comprising and active agent and a band of an insoluble material circumscribing and binding together the first and second layer. The first layer is adapted to swell in the stomach and facilitate retention therein and the active is released over a prolonged period of time.

### SUMMARY OF THE INVENTION

The present inventors have found that a solid form having two or more different zones containing a compacted fill material having a particular combination of characteristics provides a beneficial combination of delivery of the active material at acceptable dose levels and with fewer or lower quantities of excipients typically employed in capsules or tablets. The compacted fill material is less compacted than in a tablet but more than in a capsule formulation. The separation of the compacted fill material into separate zones enables aesthetic or functional characteristics to be built in to the solid form.

The invention provides in a first aspect a solid form comprising at least one film enrobing a compacted fill material wherein:
i) said compacted fill material comprises at least one active material;
ii) said solid form shows a weight loss that is less than 1% during a 30 minutes United States Pharmacopeia (hereinafter referred to as USP) friability test USP 29 Test Number 1216 (page 3046);
iii) said compacted fill material has a density of at least 0.5 g/ml based on the total solid volume of the solid form and a tensile strength of less than 0.9 MPa; and
iv) said compacted fill material comprises at least a first zone and a second zone and said active material is present in at least one of said zones.

### DETAILED DESCRIPTION OF THE INVENTION

Advantageously, by constructing the solid form with separate zones of compacted fill material, accurate dosing of the active material may be achieved so improving the uniformity of the active material from solid form to solid form. This is beneficial in quality control during manufacture.

Suitable active materials include a pharmaceutical active, food component or product, veterinary active, cosmetic component or product, an appetite suppressant, detergent component or product or nutraceutical component or product. Preferably, the solid form comprises at least one film enrobing a compacted fill material wherein the compacted fill material comprises at least one active material and at least one of a disintegrant and a wetting agent and the compacted fill material is selected from a pharmaceutical product, a food product, a veterinary product, a cosmetic, an appetite suppressant, a detergent product and a nutraceutical product, the said solid form shows a weight loss that is less than 1% during a 30 minutes United States Pharmacopeia Friability Test USP 29 Test Number 1216 (page 3046) and the said compacted fill material comprises a first zone and a second zone.

The separate zones may differ in size, shape, composition or a combination of these factors. Preferably the separate zones are in the form of separate layers in the solid form. In a preferred embodiment, the separate zones comprise 2 or more layers, each layer having a different composition from that in any other layer. The two or more zones may be separated by a physical barrier between any two or more zones or the two or more zones may not be separated by a physical barrier. Where a barrier between two or more zones is present the compacted fill materials of the two zones are not in intimate contact.

The composition in each zone may exhibit different release profiles, for example the composition in one zone may provide immediate release of the active material and the composition in a different zone may provide controlled release of the active material in that zone.

Suitably, at least one of the zones of compacted fill material comprises at least one active material provides immediate release, fast release, sustained release, delayed release, controlled release or pulsatile release. These terms are common in the art and understood by the skilled person.

The term “immediate release” is employed herein in accordance with its meaning known in the art and refers to a solid form in which the active material is released rapidly after administration. A typical release rate for an “immediate release” solid form is suitably not less than 85% drug release in 60 minutes, preferably in 45 minutes and especially in 30 minutes in the test specified in USP Edition 29 Test Number 711 at page 2673 for said active material when said active material is placed in a dissolution medium as specified in the USP dissolution specification or selected from dissolution media specified in the USP according to the solubility properties of said active material. This is referred to in the USP as “Q” time. The term “immediate release” includes “fast release”.

The solid form suitably comprises an active material which exhibits immediate release. The solid form may additionally comprise an active material which does not exhibit immediate release. If desired, the solid form may comprise an active material which exhibits immediate release and be free of an active material which does not exhibit immediate release.

The term “controlled release” as used herein refers to a solid form characterized by slower active release kinetics, compared to an immediate release solid form. The solid form preferably comprises an active material exhibiting a fast release. The solid form may comprise a further active material which does not exhibit fast release. As desired, the solid form does not contain an active material which does not exhibit a fast release.

The compacted fill material is suitably compacted during the manufacture of the solid form. The compaction process is preferably carried out at lower compaction forces than conventionally applied in producing tablets.

The compacted fill material preferably has a density of less than 1.1 g/ml and more preferably less than 1.05 g/ml. The density of the compacted fill material is suitably at least 0.55 g/ml, preferably, the density of the compacted fill material is from 0.55 to 1.04 g/ml, more preferably from 0.62 to 1.04 g/ml and desirably from 0.75 to 1 g/ml. The density of the solid form is suitably higher than that for conventional capsules and as the density contributes to the release profile of the solid form, this may be optimized by the formulator according to the release profile required.

The compacted fill material suitably has a tensile strength of less than 0.9 MPa, preferably less than 0.5 MPa, especially less than 0.2 MPa and particularly less than 0.1 MPa. The compacted fill has sufficient tensile strength to retain the physical integrity of the compacted fill and is preferably at least 0.05 MPa. The robustness of the solid form is suitably provided by the enrobing film rather than by the compacted fill material.

The solid form of the present invention has excellent robustness or physical strength. The robustness of a solid form may suitably be defined by measuring the weight loss of 10 solid forms when rotated in a USP friability apparatus. This test is as set out in USP 29 Test Number 1216 at page 3046. The solid form of the present invention shows a weight loss of less than 1% when tested for 30 minutes in a friability drum. As conventional solid forms such as coated tablets are considered to be robust when the weight loss after 4 minutes of friability testing is less than 1% measured according to USP 29 Test Number 1216 at page 3046, the solid form of the present invention is especially robust.

The density of the compacted fill material of the solid form of the present invention refers to the total weight of the fill material divided by the total volume of the solid form within the film material. This is typically referred to as the “apparent” density of the solid form. Unless otherwise stated or the context clearly requires, references to density herein are to “apparent” density.

The apparent density of a conventional tablet is typically greater than 1 g/ml as disclosed in, Pharmaceutical Technology, 27 (4), 67-80. In a conventional hard capsule, the fill material is lightly tamped so as to form a very weak slug that breaks up in the capsule shell, due to the air space above it. In a conventional hard capsule, the density of the fill material is therefore similar to the bulk density of the loose powder. The latter is typically less than 0.5 g/ml as disclosed in, Pharmaceutical Technology, 27 (4), 67-80. The density of the compacted fill material of the present invention is at least 0.5 g/ml based on the total solid form volume.

A typical method for determining the density D of the fill material in the present invention is to determine the fill weight W (1), the fill volume V, which depends on the size of the tooling used to manufacture the solid forms and to calculate D using equation (2).

W=Wt−Wf(g), where Wt is the weight of the total enrobed solid form and Wf is the weight of the film enrobing the solid form. (1)

D=W/V(g/ml) (2)

For a solid form of the present invention having a 70 microns thick film and made with oblong concave tooling of 16.6 mm length and 7.3 mm width, the volume V of the fill material is calculated using equation (3)

V=(212.7+110.8t)/1000 (ml), where t is the sidewall thickness of the solid form (mm), typically measured using a micrometer. (3)

For a tablet or compact that is made using 13 mm diameter flat round punches, the volume V of the fill material is calculated using equation (4):

V=[π(13/2)²t]/1000 (ml), where t is the tablet thickness (mm), typically measured using a micrometer. (4)

Conventional tablets generally need to be robust for subsequent processing and handling such as film coating and packaging. Such tablets are considered to be robust when the tensile strength of the compacted fill material is at least 1.0 MPa for example as disclosed in Pharmaceutical Technology, p 52-62, April 2005 (Douglas McCormick,—Evolutions in Direct Compression).

A typical method for determining the tensile strength for round flat faced cylinder shapes is to measure the crushing force (also called hardness) of compacts on a tablet hardness tester and calculate the tensile strength a using equation (5) for example as disclosed in Journal of Pharmaceutical Sciences, vol. 59 (5), 688-691 “Determination of tablet strength by the diametral-compression test”, (Fell J. T. and Newton J. M., 1970), (5) σ=2P/πDt (MPa), where P is the crushing force (N), D is the compact diameter (mm), and t is the compact thickness (mm), typically measured using a micrometer.

The compacted material in the solid form may be present in more than two zones. The same material may be present in more than one zone provided the two zones comprising the same material are separated by a further zone comprising a different material.

Preferably, the compacted fill material in the first and second zones and optionally further zones is in the form of layers within the solid form. Preferably, the compacted material is present in the solid form in two or more layers.

The solid form may be adapted such that the compacted fill material in the two or more zones have different release characteristics. The compacted fills in separate zones may be released sequentially in use for example where it is desired to release the compacted fill in each of the zones at different times or simultaneously. In a preferred embodiment, the compacted fill in the first zone is released immediately on use and the compacted fill in the second zone is released in a controlled manner.

In a preferred embodiment, one or more of the zones suitably provides immediate release or rapid dissolution of the active material. Suitably the active material comprises at least one pharmaceutical active and said at least one pharmaceutical active has a mean dissolution which meets the USP dissolution specifications specified in the test in USP Edition 29 Test Number 711 at page 2673 for said active material when said active material is placed in a dissolution medium as specified in the USP dissolution specification or selected from dissolution media specified in the USP according to the solubility properties of the active material. Where a dissolution medium is specified in the USP for an active material, this is suitably employed in the dissolution test. Where there is either:
i) no USP test for the active material;
ii) more than one test for the active material; or
iii) the active does not meet the USP specification with the specified medium;
   the skilled person will select the most appropriate medium for the dissolution test from the USP dissolution media specified in the USP having regard to the dissolution characteristics of the active material.

Examples of media in which the dissolution test may be carried out include: (i) the medium specified in the USP preferably for said at least one active material, (ii) water, (iii) 0.1 M HCl or (iv) phosphate buffer having a pH between 5.8 and 8.0.

In a preferred embodiment the compacted fill material comprises at least one active material and at least one of a super disintegrant and a wetting agent. Preferably, the at least one of the active material has a mean dissolution of at least 75% in 300 seconds in the test specified in the USP Edition 29 Test Number 711 at page 2673 for said active material when the active material is placed in a dissolution medium as specified in the USP dissolution specification or selected from dissolution media specified in the USP according to the solubility properties of the active material or as selected by the skilled person for example selected from: (i) the USP for the at least one active material, (ii) water, (iii) 0.1 M HCl or (iv) phosphate buffer having a pH between 5.8 and 8.0. A solid form meeting this dissolution test is considered herein to be a “fast release” solid form.

In a preferred embodiment, particularly where controlled release of the active material may be required, the compacted fill in one or more layers comprises a comprises particles comprising the at least one of said active material dispersed within a matrix and the active material exhibits a controlled release.

Suitably the matrix of the compacted fill material comprises a polymer. The matrix may be soluble in aqueous medium such that in use the matrix swells and then dissolves whereby the active material is released. Alternatively, the matrix may be insoluble such that in use a solvent for example water enters the matrix and on reaching particles of the active, dissolves the active material in the dissolution medium.

The compacted fill material may contain at least one material from which the matrix is formed, herein referred to as a “matrix former”. Examples of suitable hydrophilic matrix formers include hydroxypropylmethyl cellulose, sodium carboxymethyl cellulose, alginates, carrageenans, xanthan gum, locust bean gum, carbopol, guar gum, hydroxypropyl cellulose, methyl cellulose, polyethylene oxide, polymethacrylates, mannitol, polyvinyl alcohol.

The hydrophilic matrix former suitably has a viscosity in the range of 80-120,000 cPs. A 2% w/v aqueous solution of the matrix former at 20° C. is typically used to measure the viscosity.

Examples of a suitable insoluble matrix former include hydrogenated vegetable oils, microcrystalline wax and carnauba wax, ethylcellulose, polyamide, polyethylene, polyvinyl acetate, cetyl alcohol, glyceryl monostearate, glyceryl behenate, glyceryl monooleate, glyceryl palmitostearate, polacrilin potassium, stearic acid, stearyl alcohol, yellow wax, zein, hydrogenated castor oil.

In another preferred embodiment, the compacted fill in one or more layers comprises a pressure sensitive multiparticulate and at least one cushioning agent; wherein the pressure sensitive multiparticulate and/or the cushioning agent comprises at least one active material.

The term “multiparticulate” is known to those skilled in the art. As used herein, “multiparticulate has the meaning known to those killed in the art and refers to a material having discrete particles, each of which particle is itself composed of smaller particles which are bound together by physical or chemical interactions to produce the multiparticulate. Examples of multiparticulates include pellets, granules, spheres, microspheres, freeze dried material and crystals. The multiparticulate for use in the present invention may be coated or uncoated. Multiparticulates can have any shape and texture and can be produced by known processes. When taken orally, the multiparticulate suitably disperses freely in the gastrointestinal tract, optimizes absorption, and can minimize side effects. A multiparticulate may contain one or more components.

As used herein, the term “pressure sensitive multiparticulate” means a multiparticulate that has a physical attribute or characteristic for example its rate of dissolution, efficacy, or mechanical strength altered detrimentally to a material extent when the multiparticulate is compacted as compared to the uncompacted multiparticulate. Appropriate tests to determine whether an attribute or characteristic has been detrimentally affected as a result of compaction of the multiparticulate will depend on the particular characteristic being measured and are known to the skilled person.

In a preferred embodiment, the solid form includes a ‘disintegrating layer’ of compacted fill material comprising a disintegrant or comprising a material acting as a disintegrant.

In conventional capsules and tablets, the disintegrant is added to the active ingredient to facilitate rapid breakup of the solid form. In conventional tablets and capsules, the disintegrant is typically present as a component in a blend with the active material. In these products, disintegration typically occurs through wicking, swelling and deformation. Disintegration may be in the form of rapid break up of the conventional solid form or may occur through a slow eroding process.

The disintegrating layer may be disposed between a separate zone or layer of compacted material comprising an active material and the water-soluble film to adequately separate the compacted fill from the enrobing film.

Without wishing to be bound by any theory in the present invention, where the disintegrant zone is located between the compacted fill material and the enrobing film, the separate disintegrant zone or layer is believed to separate the enrobing film from the compacted fill material so exposing the compacted fill to the dissolution media. The result is a reduction in disintegration time which is beneficial for immediate release solid forms and especially fast release solid forms.

In another preferred embodiment, the solid form comprises a first compacted fill material in a first zone, a second compacted fill material in a second zone and a separating layer between the compacted fill materials in the first and second zones so as to keep the first and second fill materials separate until use.

Advantageously, the provision of two or more layers of compacted fill material permits the compacted fill material in each of the layers to comprise different components. Where the components in the different layers are incompatible, the layers may be separated by a further layer between the first and second layer.

The separate zones or layers may contain different active materials whereby the solid form comprises a plurality of active materials.

As desired, the disintegrating layer may be interposed between two zones of compacted fill material to separate the compacted fill layers and to aid dissolution of the compacted fill material.

Advantageously, provision of a separate disintegrating zone or layer avoids having to blend the disintegrant with the active material so reducing the number of manufacturing process steps.

In addition to the at least two zones of compacted fill material, other zones, preferably at least one layer of a non-compacted fill material may be present. In a preferred embodiment, the solid form comprises a compacted fill material in a first zone and a second zone and a further zone comprising a non-compacted fill material. The further zone is suitably interposed between the first and second zones so as to provide a physical barrier between the compacted fill in the first and second zones. The compacted fill in the first and second zones may be the same or different as desired.

The non-compacted zone suitably comprises a material that exhibits a melting transition, i.e. turns from solid at room temperature to a liquid upon heating and then returns to a solid upon cooling to room temperature. Preferably, the material exhibits a clear melting transition at temperatures below 100° C., more preferably below 60° C., to enable rapid solidification upon layering over the first compacted zone. However with the use of appropriate processing equipment for example an extruder, higher melting point materials may also be used.

Examples of suitable materials to form the non-compacted zone include polyethylene glycol, polyethylene oxide, polymethacrylates, polyvinyl alcohol, stearic acid, cetyl alcohol, hydrogenated oils, glyceryl behenate, glyceryl palmitostarate, glyceryl monostearate, waxes, and heat stable sugar alcohols.

The non-compacted fill material in the non-compacted zone suitably comprises a film, made prior to the manufacture of the solid form of the present invention.

Desirably at least one of the layers comprises an active material and at least another layer comprises another active material.

The compacted fill material is suitably present in said solid form in at least three layers, optionally at least four and, as desired, at least five layers.

The compacted fill material in one of the zones may have the function of aiding manufacture of the solid form. For example, where the compacted fill material in the first zone has adhesive characteristics, a layer of a second material may be applied to the first material so as to reduce the risk of process complications during production.

In a preferred embodiment at least one of the zones of compacted fill material is a processing aid layer which suitably provides a uniform or smooth surface on which to place a further layer of fill material or the enrobing film. An anti-sticking layer to prevent sticking of, for instance, the active layer to processing equipment, for example a compaction punch, a cushioning layer to prevent damage of a pressure sensitive layer or a bulking layer which suitably provides volume to the solid form when active present in a very low amount may be employed as one of the layers.

At least one of the zones of compacted fill material may be a separating layer, disintegrant layer, aesthetic layer to enhance visual appeal of the solid form or to aid the user in complying with a dosage regime, or stability enhancing layer.

In a preferred embodiment, the compacted fill material comprises a low dose active material layer and a bulking layer in intimate contact with the active layer.

In a further embodiment, the compacted fill material comprises a low dose active layer entrapped between two bulking layers.

The film enrobing the compacted fill material is preferably a water-soluble film.

Desirably, the film is in intimate contact with the compacted fill material. By “intimate contact” it is meant that the film and the compacted fill material or compacted fill material in the at least first and second zones are in direct contact preferably over the entire surface although some areas not being in direct contact may be acceptable.

Suitably, the compacted fill material comprises at least one active material and at least one of a disintegrant and a wetting agent.

In a preferred embodiment the compacted fill material comprises at least one active material and at least one of a super disintegrant and a wetting agent and wherein the at least one of the active material has a mean dissolution of at least 75% in 300 seconds in the test specified in the USP Edition 29 Test Number 711 at page 2673 for said active material when the active material is placed in a dissolution medium selected from the USP for the at least one active material.

The invention in a further aspect provides for the use of a solid form according to the invention in a method of treatment of the human or animal body by therapy. The invention also provides for the use of the solid form in the manufacture of a medicament for a method of treatment of the human or animal body by therapy.

Advantageously, solid forms according to the present invention in which the compacted fill is enrobed in a film provide immediate release or delivery of the active material.

The compacted fill material suitably comprises a disintegrant. Examples of suitable disintegrants include alginic acid, calcium phosphate, carboxymethylcelluloses, powdered cellulose, chitosan, colloidal silicon dioxide, guar gum, magnesium aluminium silicate, methylcellulose, microcrystalline cellulose, povidone, sodium alginate, starch, pregelatinised starch.

Super disintegrants are a type of disintegrant. In a fast release solid form, disintegrating layer or zone, the compacted fill material suitably comprises a super-disintegrant. The class of materials referred to as “super disintegrants” are known in the art and generally refer to such materials as crosslinked celluloses, crosslinked starches and crosslinked polymers. Examples of such include croscarmellose sodium, sodium starch glycolate, polyvinyl pyrrolidone, crospovidone, or low substituted hydroxypropyl cellulose.

The disintegrant may be used in an amount of 0.1 to 25% by weight of the compacted fill material, more particularly, 5 to 15% by weight especially 8 to 12% by weight, for example 10% by weight of the compacted fill material. The particular amount of disintegrant will be selected according to the particular disintegrant, formulation and use.

The super disintegrant may be used in an amount of 0.1 to 10% by weight of the compacted fill material, more particularly, 0.25 to 6% by weight, especially 1 to 4% by weight of the compacted fill material.

Wetting agents may also be used in the compacted fill material of the present invention. The class of materials referred to as “wetting agents” are well known in the art and generally refer to such materials that are usually surface-active materials or surfactants, which reduce the contact angle between solid and liquid and therefore increase the adhesion of the liquid to the solid surface of an active material. Examples of such include hypromellose, docusate sodium, sodium lauryl sulfate, polyoxyethylene sorbitan fatty acid esters, sorbitan esters, polyoxyethylene alkyl ethers, dioctyl calcium sulfosuccinate. Other examples of wetting agents include solubilizing agents such as povidone, cyclodextrins, poloxamers, glyceryl monostearate.

The wetting agent is suitably used in an amount of 0.01 to 10% by weight of the compacted fill material, more particularly, 0.1 to 2% by weight.

Suitably, the active material has a solubility in water of 1 g in less than 1 g water, 1 g in 1 to 10 g water, 1 g active in 10 to 30 g water, 1 g active in 30 to 100 g water, 1 g active in 100 to 1,000 g water, 1 g active in 1,000 to 10,000 g water, and 1 g active in more than 10,000 g water.

Any solid active material having the above water solubilities may be used in the present invention alone or in combination.

Examples of suitable classes of pharmaceutical actives include an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active, motion sickness active, anti-HIV, anti-malaria actives, anti-cholesterol actives, respiratory actives, gastronintestinal actives, cardiovascular actives, antidiabetes actives, central nervous system actives, anti-infection actives, mucolytics, proton pump inhibitor and nasal decongestants.

Examples of suitable actives include paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, pheneizine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocamide, tinidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceutically or nutraceutically acceptable salts, acids, esters, isomers, and metabolites thereof.

Where more than one active material is present, the two or more actives may be from the same class or may be from different classes. Examples of combinations of active materials from different classes include an antibiotic in combination with one of a decongestant, an anti-inflammatory, a cough expectorant, a cough suppressant or an active for treatment of cough or common cold, a proton pump inhibitor; anti-hypertension and anti-cholesterol actives.

Examples of classes where two or more active materials from one class may suitably be employed include respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants, anti-HIV, anti-malaria actives.

Examples of particular combinations of active materials include: Paracetamol and Caffeine; Aspirin and paracetamol; Paracetamol and pseudoephedrine; Paracetamol and phenylephrine; Ibuprofen and codeine; Ibuprofen and pseudoephedrine; Paracetamol and diphenhydramine; Acravistine and pseudoephedrine; Paracetamol and dextromethorphan; Parcetamol and guaphenesin; Paracetamol, caffeine, aspirin; Aspirin and caffeine; Zidovudine, lamivudine and abacavir; Pravastatin and aspirin; Lamivudine and zidovudine; Roziglitazone and Metformin; Ezetimibe and fenofibrate; Amoxicillin and Clavulanate; Trimetoprim and sulfamethoxazole; Amlodipine and benazepril; Valsartan and Hydrochlorothiazide; Irbesartan and Hydrochlorothiazide; Losartan and Hydrochlorothiazide; Fenofibrate and Metformin; Abacavir and lamivudine; Dutasteride and tamsolusin; Atazanavir and ritonavir; Ritonavir and Saquinavir; Propoxyphene and paracetamol; Hydrocodone and paracetamol; tramadol and paracetamol; Metocarbamol and paracetamol; Memantine and Donepezil; Glyburide and Metformin; Pioglytazone and Metformin; Rosiglitazone and Glimepiride, Benazepril and Hydrochlorothiazide; Atorvastatin and Torcetrapib; Eprosartan and Hydrochlorothiazide; Amlodipine and Atorvastatin; Ezetimibe and Simvastatin; Telmisartan and Hydrochlorothiazide; Olmesartan and Hydrochlorothiazide; Lopinavir and Ritonavir; Emtricitabine and Tenofovir; Fosamprenavir and Ritonavir; Amprenavir and Ritonavir; Tipranavir and Ritonavir; Atovaquone and Proguanil; Lansoprazole, Amoxicillin and Clarithromycin; Lansoprazole and Naproxen; 5-aminosalicylic acid, 4-aminophthalic acid; Clarithromycin, Ranitidine and Bismuth citrate; Bismuth subsalicylate, Metronidazole and Tetracycline; Montelukast and Loratadine; Fexofenadine and pseudoephedrine; Guaifenesin and pseudoephedrine.

Low levels of active material, for example from 1 to 30% may be employed as desired in a zone or layer. However, the amount of the active material present in the compacted fill material in a zone or layer is suitably at least 30% by weight of the compacted fill material in that zone or layer, and particularly at least 70% of the compacted fill material. In an embodiment in which a high dose of active material is required, the amount of active material is desirably at least 90% and especially at least 95%. In some applications, the active material may be at least 99%. The total level of active material in the solid form is suitably selected according to the active or combination of actives and the intended use. In a preferred embodiment, the solid form may in total contain at least one active material in an amount greater than 100 mg, desirably greater than 300 mg. In a particularly preferred embodiment, the total active material content of the solid form is from 400 mg to 800 mg. The at least one active material may be disposed in the separate layers in any desired proportions, depending on the application and the particular at least one active material.

For low dose active materials, the solid form may contain the active material in an amount of less than 100 mg, less than 50 mg, less than 10 mg, less than 1 mg, or less than 0.1 mg, or less than 0.05 mg. Preferably at least one zone, preferably layer, comprises the active material at a level of at least 100 mg. At least one zone may comprise the active material at a level of less than 100 mg, preferably less than 50 mg.

The active material of the present invention is preferably a powder and this suitably includes such powders as granules, micronized powders, spray-dried powders, freeze-dried powders and pellets.

The compacted fill material of the present invention may contain at least one filler. Examples of the filler include a broad category of excipients such as glidants, binders and lubricants. Examples include microcrystalline cellulose, dicalcium phosphate, lactose, calcium carbonate, calcium phosphate dibasic anhydrous, calcium phosphate dibasic dehydrate, calcium phosphate tribasic, powdered cellulose, silicified microcrystalline cellulose, cellulose acetate, compressible sugar, confectioners sugar, dextrin, dextrose, ethylcellulose, fructose, lactitol, starch, pregelatinized starch, sucrose, talc, xylitol, maltodextrin, magnesium carbonate, maltose, mannitol, polydextrose, sodium alginate, sodium chloride, sorbitol, sucrose, sugar spheres, acacia, carrageenan, carbomer, chitosan, hydroxypropylmethylcellulose, carboxymethylcellulose sodium, gelatin, guar gum, hydroxyethyl cellulose, hydroxypropyl cellulose, methylcellulose, povidone, zein, citric acid, sodium bicarbonate, alginic acid, carboxymethylcellulose calcium, colloidal silicon dioxide, low substituted hydroxypropyl cellulose.

When filler is present, the compacted fill material may comprise in excess of 70%, more than 90% and possibly at least 99% by weight of filler where low dose active solid forms are required. Alternatively, the filler may be present in an amount of less than 70% by weight of the compacted fill material, preferably less than 30% by weight, for example from 1 to 10% by weight and from 1 to 5% by weight of the compacted fill material. As desired, the enrobed solid form of the present invention may contain no filler in the compacted fill material.

Known processes for making oral solid forms such as the unit processes typically required to produce tablets and capsules, typically involving blending, may lead to components of the product segregating during the blending process causing poor content uniformity. A granulation step may be required to produce a uniform blend for tablet manufacture although this may add complexity and cost.

The inventors have now developed a method of making a solid form which enables the direct deposition of very small amounts of pure active materials by layering and deposition of a fill material comprising an active material directly into the solid form so avoiding blending and granulation steps to produce the final coated solid form in a single continuous process.

A further aspect of the invention provides a method of making a solid form comprising at least one film enrobing a compacted fill material comprising:
i) providing a first film shaped to define an interior volume for holding said compacted fill material and having an open end;
ii) depositing via the open end a first zone of fill material to be compacted in the interior volume and optionally applying pressure to the fill material so as to compact the first zone of fill material;
iii) depositing a second zone of fill material to be compacted in the interior volume such that the interior volume comprises two zones of fill material wherein at least one of the fill material in the first or second zone comprises an active material;
iv) applying pressure to the fill material so as to compact the at least second zone of fill material so forming the said compacted fill material;
v) applying a second film over the said open end to close the said open end; and
vi) sealing the first and second film together to enrobe the compacted fill material and provide the solid form.

The invention also provides a method of making a solid form comprising at least one film enrobing a compacted fill comprising:
i) providing a first film shaped to define an interior volume for holding a compacted fill material and having an open end;
ii) depositing via the open end a first zone of fill material in the interior volume;
iii) depositing a second zone of fill material in the interior volume such that the total interior volume comprises two zones of fill material wherein the first and/or second fill material comprise an active material;
iv) applying pressure to the fill material so as to compact the at least two zones of fill material
v) applying a second film over the said open end to close the said open end; and
vi) sealing the first and second film together to enrobe the compacted fill material and provide the solid form.

Suitably the zones comprising the compacted fill material are in the form of layers. The compacted fill material in the different zones or layers may be the same as in other zones or layers but is preferably different to that in other zones or layers. Each layer of fill material may be compacted prior to depositing the next layer of fill material, several layers may be deposited and then compacted together or all the layers may be compacted in a single compaction step. The at least two layers may be deposited in a single step or each or several layers layer may be deposited in a separate step. The layers may be deposited using a volumetric system, a vacuum system, dosing pump or syringe.

High dose level active materials may require the addition of binders to give a final solid form of adequate integral strength to withstand subsequent processes such as coating and packaging in conventional solid forms whilst keeping the solid form at an acceptable size. A further advantage of the present invention is that the level of binder may be reduced as the enrobing film provides adequate strength to withstand subsequent processes. The layered solid form of the present invention advantageously avoids or reduces processing or product drawbacks such as sticking and “picking” where powder remnants of the solid fill material stay attached to the compaction apparatus which may create irregular topography of the surface of subsequently processed solid forms.

A further advantage from employing separate zones of compacted fill material includes the ability to optimize the solid form for therapeutic benefits. By manipulation of the active formulations within the layer or by using different sequences of layers or by varying the excipients within non active containing layers it is possible to produce solid forms with tailored release profiles for example a layer with immediate release and a further layer with a controlled release profile. By tailoring the release profile of the layers, compliance with a dosage regime for the user may be improved. The inclusion of different active materials in separate layers within the solid form also gives the possibility of therapeutic benefits not currently achievable using traditional tableting processes for example pharmacological synergy between two actives generated by the combination of different layers in the solid form and, for example increased patient compliance by decreasing the number of solid forms to take daily.

The enrobed solid form of the present invention comprises a film enrobing a compacted fill material wherein the compacted fill material is present in at least two zones, preferably layers, more preferably, at least three layers, particularly, at least four layers, more particularly, at least five layers.

Forms other than layers for the compacted fill are within the scope of the invention and include for example a first zone defined by a granule and a second zone of a fill material around the granules so providing “islands” of granules within a “sea” of other fill material such that discrete zones of different fill material are provided.

The film to be used to enrobe the present invention may be any film capable of enrobing the compacted fill material without adversely impacting the desired dissolution profile. The film to be used may comprise water soluble components, water insoluble components or may comprise soluble and insoluble components in combination.

Preferably, the compacted fill material of the present invention is enrobed by a film comprising at least one water soluble polymer. Films generally useful in the present invention include those that are thermo formable and generally have dissolution rates appropriate for the preparation of rapid release, preferably immediate release, solid forms of the invention. Examples of such water soluble polymers include cellulosic materials such as hydroxyethyl cellulose, hydroxypropyl cellulose, hydroxypropyl methyl cellulose; polyvinyl alcohol; hydrocolloids such as carrageenan, alginate and pectin; and water soluble acrylates. Examples of water insoluble polymers include ethylcellulose, methacrylates and cellulose acetate. The films used in the invention may be gelatin free. The films may contain plasticizers such as lactic acid, citric acid, polyethylene glycol, sorbitol, glycerine, triethylcitrate, propylene glycol, phthalates, triglycerides, triacetin, tributylcitrate, etc. WO 2004/026284, WO 02/083779 and WO 03/095548 disclose further examples of films that may be used in the invention and such are incorporated herein by reference. Examples of films that may be used in the present invention are available under the trade name XGEL UNO from BioTec Films LLC, Tampa, Fla., US. Films for use in the present invention may be made in a conventional manner. If desired, an adhesive and use thereof can be used to aid in sealing the films together. Suitable adhesive compositions include those set forth in WO 04/10337 and WO 04/103338—both of which are incorporated herein by reference.

The solid forms of the present invention may be enrobed and prepared in accordance with the methods disclosed in WO 03/096963, WO 05/030115, WO 05/030116 and PCT/GB2005/001077—all of which are incorporated herein by reference.

### DESCRIPTION OF THE DRAWINGS

The invention is illustrated by reference to the accompanying drawings in which:

FIG. 1 shows a cross section of a solid form according to the present invention;

FIG. 2 shows a cross section of a solid form according to the present invention;

FIG. 3 shows a series of cross sections of a solid form in different states of production produced according top the method of the present invention; and

FIG. 4 shows a plot of the percentage release versus time of an active material from a solid form according to the present invention.

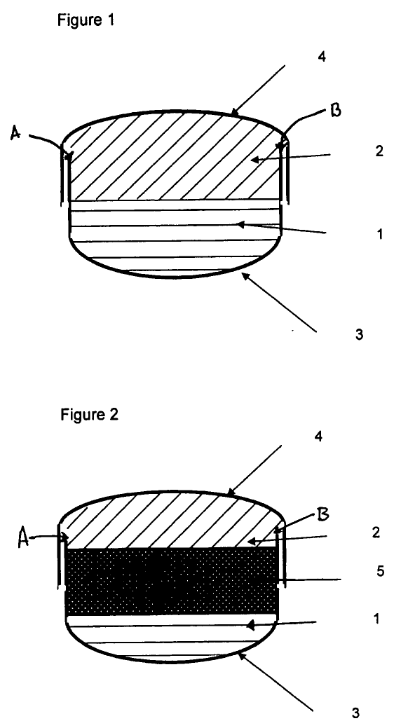

In FIG. 1, the solid form contains two layers (1) and (2) of compacted fill material. The compacted fill material in the two layers may be of different formulation or physical characteristics as desired. The layers (1) and (2) are located in a film (3) which has an open end between the points A and B and are arranged parallel to the plane of the opening between the points A and B. A second film (4) is located over the open end of film (3) so as to close it and suitably seal the overlapping films (1) and (2) within the solid form. The layers (1) and (2) are in intimate contact with one another and it is possible that some mixing of the layers may occur at the interface. At least one of the layers (1) and (2) contains an active material, preferably a powder and the compacted fill material which comprises the layers (1) and (2), has a density of at least 0.5 g/ml based on the total solid volume of the solid form and a tensile strength less than 0.9 MPa. The solid form shows a weight loss that is less than 1% during a 30 minutes USP friability test.

In FIG. 2, a third layer (5) is included between the bottom layer (1) and upper layer (2). The middle layer (5) may be employed to act as a physical barrier between the two layers (1) and (2) or may contain components which provide functional benefits in addition to physically separating the layers (1) and (2). At least one of the layers (1), (2) and (5) contains an active material, preferably a powder and the compacted fill material which comprises the layers (1), (2) and (5), has a density of at least 0.5 g/ml based on the total solid volume of the solid form and a tensile strength less than 0.9 MPa. The solid form shows a weight loss that is less than 1% during a 30 minutes USP friability test.

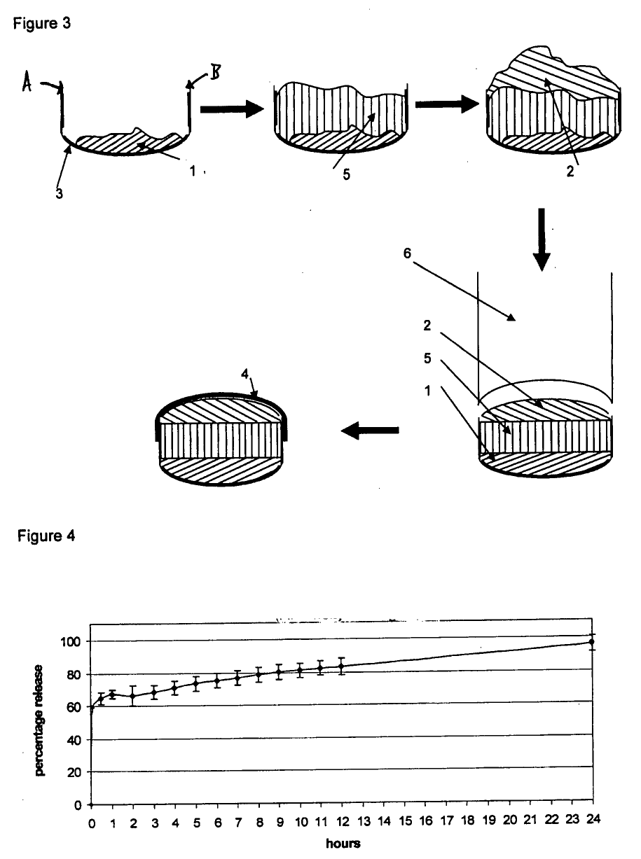

In FIG. 3, the first layer of fill material (1) is dosed to the film (3) through the open end between points A and B, the second dose of fill material (5) is then dosed on top of the first fill material (1), a third fill material (2) is then dosed onto the second fill material (5). The film (3) containing the three doses of fill material (1), (2) and (5) is then subjected to compression, suitably by a compression punch (6) to increase the density of the compacted fill materials (1), (2) and (5) to at least 0.5 g/ml based on the total solid form of the solid form. The second film (4) is then applied across the opening between points A and B to close and so enrobe the compacted fill material to provide the final solid form.

The invention is described with reference to the following illustrative examples. In this specification, all parts and percentages are by weight unless otherwise noted.

### EXAMPLES

### Materials

| Material | Grade | Supplier |
|---|---|---|
| Ibuprofen | BP/EP grade, mean | Shasun Chemicals & Drugs |
| | particle size: 67.5 microns | Ltd |
| Hydrochlorothiazide | | Spectrum Chemicals |
| Microcrystalline cellulose | Avicel ® PH 200 | FMC Corp, Philadelphia PA |
| Metformin HCl | EP grade | s.a. Pharminnova B.V. |
| croscarmellose sodium | AC-DI-SOL ® | FMC Corp, Philadelphia PA |
| Microcrystalline cellulose | Avicel ® PH 102 | FMC Corp, Philadelphia PA |
| Polyethylene glycol | NF, 1000 | Union Carbide |
| Polyethylene glycol | NF, 8000 | Union Carbide |
| FD&C Red #40 AL | | Sensient |
| FD&C Blue #2 AL | | Sensient |
| FD&C Blue #1 AL | | Sensient |
| FD&C Yellow #6 AL | | Sensient |
| Non-pareils, sugar | | Chr Hansen |
| spheres | | |
| Sodium Starch Glycolate | Explotab ® | Mendell |
| Sucrose | Fine granular | United Sugar |
| Crospovidone | | ISP |
| Theophylline | USP grade | Shandong Xinhua |
| | | Pharmaceutical Co., Ltd. |
| benzyl alcohol | | EM Science |
| hypromellose (HPMC) | Methocel ® E 15LV | Dow Chemical |
| Triacetin | | Eastman |
| Hypromellose (HPMC) | Methocel ® K 4 M | Dow Chemical |

### Methods

Fill material: The ibuprofen and theohylline active powders were sieved through a 24 mesh screen (710 microns) prior to weighing. Powders were weighed out and blended in a Speedmixer DAC150FVZ-K for 5 seconds at 3000 rpm. The powder fill material was stored in a plastic bottle or double plastic bags until use. Ibuprofen is practically insoluble in water (1 g in more than 10,000 g of water), theophylline is slightly soluble in water (1 g in 100-1000 g water), metformin hydrochloride is freely soluble in water (1 g in 1-10 g water), hydrochlorothiazide is very slightly soluble in water (1 g in 1000-10000 g water).

Enrobed solid form: Soluble films known as XGEL UNO and supplied by BioTec Films LLC were cut into strips 6 centimeters by 20 centimeters approximately. The lower and upper films had a thickness of about 120 microns. The lower film was heated sufficiently to thermoform under vacuum into dose cups about 3 millimeters in height to conform to cavities (7.5 millimeters width by 16.75 length millimeters) with the cavity depth determined by height-adjustable dose-shaped lower pistons within the stainless steel die. The film strip was placed over the die and brought in contact with a heated TEFLON® coated surface by means of upward vacuum. The film was then drawn into the stainless steel die cavities by inverting the vacuum to form a strip of twelve thermoformed dose cups with 3.0 millimeters separation between adjacent dose cups. Some unused portion of the filmstrip was cut and removed. The fill composition was dosed (by volume) into the dose cups, though a paper funnel. The multiple doses were filled one on top of the others so as to form horizontal layers. Then the layered fill was lightly compacted in the dose cups with upper pistons, and the lower film was cut to separate the individual solid forms. The solid forms were then lifted by the lower pistons to expose a portion of the solid form sidewalls for application of the upper film to complete the enrobing of the solid form. An adhesive composed of 5% Methocel E15LV Premium, 45% Benzyl alcohol and 50% Triacetin, was applied (by transfer roller) to the upper filmstrip on the side to be pressed against the exterior sidewall of the dose cup. The upper film was placed over the solid forms containing the compressed layered powder fill and the film was heated by contact with the heating element using upper vacuum. The heated upper film was formed around the solid forms using the lower vacuum enclosing the fill material within the solid form by overlapping the upper film onto the sidewall of the solid forms. The top film was cut to separate the completed enrobed solid forms and the unused film was removed. The solid forms were further sealed by forcing them through a heated die under low pressure so that the cut film overlapping the sides was pressed smooth. All examples below used the apparatus set forth in WO 2005/030115,

Soluble HPMC containing films were used to enrobe the solid forms.

Dissolution was according to USP 29 with dissolution apparatus 2, paddles or 1, baskets. Disintegration testing was carried out according to USP 29

### Example 1

The powder fill was layered with the following materials: a 10 mg dose of pure Ibuprofen (used as a model for low dose insoluble active material), either covered by a top bulking layer of 320 mg of Avicel PH200 (1-1) as in FIG. 1 or entrapped between a top bulking layer of 150 mg of Avicel PH200 and a bottom bulking layer of 150 mg of Avicel PH200 (1-2) as in FIG. 2 and a 25 mg of pure Hydrochlorothiazide, either covered by a top bulking layer of 350 mg of Avicel PH200 (1-3) as in FIG. 1 or entrapped between a top bulking layer of 150 mg of Avicel PH200 and a bottom bulking layer of 150 mg of Avicel PH200 (1-4) as in FIG. 2.

Table I shows the mean weights of the solid forms and their components (the fill materials), the ibuprofen release in the dissolution test at 37° C. according to USP 29 for Ibuprofen immediate release tablets using 900 ml of phosphate buffer at pH 7.2 in dissolution apparatus 2, paddles, the hydrochlorothiazide release in the dissolution test at 37° C. according to USP 29 for hydrochlorothiazide immediate release tablets using 900 ml of HCl 0.1N in dissolution apparatus 1, baskets. USP specifications for Ibuprofen tablets for immediate release are: not less than 85% of the drug dissolved after 60 minutes (Q). This is referred to as the “Q-time.” USP specifications for hydrochlorothiazide for immediate release are: not less than 65% of the drug dissolved after 60 minutes (Q). This is referred to as the “Q-time.”

The release of the low dose of ibuprofen from the enrobed solid forms of the present invention satisfied the USP specifications for Ibuprofen tablets for immediate release. The release of the ibuprofen was significantly faster from the enrobed solid form containing 3 layers (ibuprofen in middle layer) than from the enrobed solid form containing 2 layers (ibuprofen in the bottom layer).

The release of the low dose of hydrochlorothiazide from the enrobed solid forms of the present invention satisfied the USP specifications for hydrochlorothiazide tablets for immediate release.

TABLE I
Ibuprofen and Hydrochlorothiazide release from layered enrobed solid forms of
the present invention

| | Enrobed Solid form | | | |
|---|---|---|---|---|
| | 1-1 | 1-2 | 1-3 | 1-4 |
| Number of fill layers | 2 | 3 | 2 | 3 |
| Drug | Ibuprofen | Ibuprofen | Hydrochlorothiazide | Hydrochlorothiazide |
| Position of Drug | Bottom | Middle | Bottom | Middle |
| layer | | | | |
| Drug loading (%) | 2.7 | 2.8 | 6.0 | 6.8 |
| Solid form weight | 365 | 350 | 405 | 358 |
| (milligrams) | | | | |
| Fill weight | 325 | 310 | 365 | 318 |
| (milligrams) | | | | |
| Drug release (%) | 50 +/− 5 | 97 +/− 16 | 78 +/− 3 | 84 +/− 5 |
| after 5 minutes | | | | |
| Drug release (%) | 97 +/− 2 | Nm* | 99 +/− 2 | 105 +/− 4 |
| at Q time | | | | |

*Not measured

### Example 2

The powder fill materials were filled in the following way: 390 mg of pure polyethylene glycol (PEG), used as a model for erodible, non-disintegrating, powder fill material, was filled into example (2-1); a first bottom layer was filled using 390 mg of pure (PEG) covered by a second top layer of 50 mg of a blend of Mannitol and blue pigment, as in FIG. 1, in example (2-2); a first bottom layer was filled using 390 mg of pure PEG covered by a second top layer of 52 mg of pure Crospovidone, as in FIG. 1, in example (2-3); a first bottom layer was filled using 390 mg of pure PEG covered by a second top layer of 50 mg of a blend of Sodium Starch Glycolate, Avicel PH102 and blue pigment, as in FIG. 1, in example (2-4); a first bottom layer was filled using 391 mg of pure PEG covered by a second top layer of 50 mg of a blend of Avicel PH102 and red pigment, as in FIG. 1, in example (2-5); a first bottom layer was filled using 390 mg of pure PEG covered by a second top layer of 50 mg of a blend of Sodium Starch Glycolate and blue pigment, as in FIG. 1, in example (2-6); one layer was filled using 460 mg of a blend of PEG, Crospovidone, Avicel PH102 and blue pigment in example (2-7); a first bottom layer was filled using 390 mg of pure PEG covered by a second top layer of 145 mg of sugar spheres, as in FIG. 1, in example (2-8); a first bottom layer was filled using 390 mg of pure PEG covered by a second top layer of 50 mg of a blend of Crospovidone, Avicel PH102 and blue pigment, as in FIG. 1, in example (2-9).

Table II shows the disintegration results of the solid forms, according to USP 29. FIG. 6 is showing the effect of layering and layer type on the disintegration time of the solid forms.

Examples containing disintegrant as a blend (2-7) or in the form of a layer (2-2, 2-3, 2-4, 2-5, 2-6, 2-8 and 2-9) were observed to disintegrate significantly faster than examples containing pure PEG. Examples containing PEG blended with a disintegrant (2-7). Disintegrating layers contained in examples 2-8 and 2-9 were found to decrease disintegration time at an optimal level. In example 2-8, unlike conventional disintegrants, beads are not considered disintegrants. However, placing a layer of beads between the PEG layer and the enrobing film accelerated the compact disintegration. It is believed that the beads provide a channel for the disintegration media to enter between the enrobing film and the compacted fill material thus allowing the film to fall away from the compacted fill material and disintegrate. In example 2-9, avicel and crospovidone are known as effective disintegrants for conventional solid forms. However, in this application it was found they work together in synergy effectively pushing the film away from the compacted fill material thus allowing the fill disintegration to occur. In contrast, single layers of either ingredient did not provide as rapid disintegration.

Examples 2-2, 2-3, 2-4, 2-5, 2-6, 2-8 and 2-9 are within the scope of the invention, example 2.1 and 2-7 are comparative examples.

TABLE II
PEG powder fill disintegration from layered enrobed solid forms of the present
invention and non-layered solid forms

| | | | | | | | | | |
|---|---|---|---|---|---|---|---|---|---|
| Enrobed Solid form | 2-1 | 2-2 | 2-3 | 2-4 | 2-5 | 2-6 | 2-7 | 2-8 | 2-9 |
| Number of fill layers | 1 | 2 | 2 | 2 | 2 | 2 | 1 | 2 | 23 |
| Model powder layer | PEG | PEG | PEG | PEG | PEG | PEG | PEG | PEG | PEG |
| Position of disintegrating | None | Top | Top | Top | Top | Top | None | Top | Top |
| layer | | | | | | | | | |
| Solid form weight (milligrams) | 428 | 472 | 472 | 472 | 469 | 472 | 506 | 567 | 472 |
| Fill weight (milligrams) | 390 | 440 | 442 | 440 | 441 | 440 | 460 | 535 | 440 |
| Disintegration time (minutes) | 19 | 13 | 13 | 11 | 10 | 10 | 9 | 6 | 5 |

### Example 3

The powder fill was layered with the following materials: a 362 mg dose of pure Metformin HCl entrapped between a top and a bottom disintegration layers of 25 mg of Ac-Di-Sol (3-1) as in FIG. 2 or a 312 mg of pure Metformin HCl, either entrapped between a top and a bottom disintegration layers made of 50 mg of a blend of 92% Avicel PH102 and 8% Ac-Di-Sol (3-2) as in FIG. 2 or entrapped between a top and a bottom disintegration layers made of 50 mg of Avicel PH102 (3-3) as in FIG. 2.

Table III shows the mean weights of the solid forms and their components (the fill materials), the Metformin HCl release in the dissolution test at 37° C. according to USP 29 for Metformin HCl immediate release tablets using 900 ml of HCl 0.1N in dissolution apparatus 1, baskets. USP specifications for Metformin HCl tablets for immediate release are: not less than 85% of the drug dissolved after 30 minutes (Q). This is referred to as the “Q-time.”

The release of Metformin HCl from the enrobed solid forms of the present invention satisfied the USP specifications for Metformin HCl tablets for immediate release.

TABLE III
Metformin HCl release from layered enrobed solid forms of the present
invention

| | Enrobed Solid form | | |
|---|---|---|---|
| | 3-1 | 3-2 | 3-3 |
| Number of fill layers | 3 | 3 | 3 |
| Drug layer | Metformin HCl | Metformin HCl | Metformin HCl |
| Position of Drug | Middle | Middle | Middle |
| layer | | | |
| Drug loading (%) | 80 | 69 | 69 |
| Solid form weight | 454 | 484 | 464 |
| (milligrams) | | | |
| Fill weight | 414 | 444 | 424 |
| (milligrams) | | | |
| Drug release (%) | 99 +/− 1 | 90 +/− 13 | 98 +/− 1 |
| after 5 minutes | | | |
| Drug release (%) at | 99 +/− 1 | 97 +/− 4 | 97 +/− 3 |
| Q time | | | |

### Example 4

The powder fill was layered with the following materials: a 240 mg bottom layer of a blend made of 98% Theophylline and 2% Ac-Di-Sol (immediate release layer) covered by a 238 mg top layer of a blend made of 60% Theophylline and 40% Methocel K₄M (controlled release layer) (4) as in FIG. 1.

Table IV shows the mean weights of the solid form and its components (the fill materials) and the Theophylline release in the dissolution test at 37° C. The solid forms were tested using 900 ml of simulated gastric fluid without enzymes according to USP 29 for 1 hour and 900 ml of simulated intestinal fluid without enzymes according to USP 29 thereafter in dissolution apparatus 1, baskets.

The release of Theophylline from the enrobed solid forms of the present invention was immediate for the first 5 minutes and was prolonged thereafter reaching a full release after 24 hours of dissolution. The drug release after the first hour of dissolution shows a 0.96 correlation coefficient when a straight line is passed through the data points, which defines a zero order type of release.

The full release curve plotting the percentage of theophylline released from a solid form having a layer comprising 98% theophylline and 2% ADS and a layer of 60% theophylline and 40 HPC K₄M over time of Example 4 is shown in FIG. 4.

TABLE IV
Theophylline release from layered enrobed solid forms of the present
invention

| | Enrobed Solid form |
|---|---|
| | 4 |
| Number of fill layers | 2 |
| Drug | Theophylline |
| Immediate release Drug layer | Bottom |
| Controlled release Drug layer | Top |
| Total Drug loading (%) | 73 |
| Solid form weight (milligrams) | 532 |
| Fill weight (milligrams) | 492 |
| Drug release (%) after 5 minutes | 60 +/− 2 |
| Drug release (%) after 1 hour | 68 +/− 2 |
| Drug release (%) after 6 hours | 75 +/− 1 |
| Drug release (%) after 12 hours | 84 +/− 2 |
| Drug release (%) after 24 hours | 96 +/− 3 |
| Equation of trendline from 1 hour through 24 hours | y = 1.3391x + 66.6 |
| data points | |
| R² value of trendline | 0.96 |

### Example 5

A powder fill was layered with the following materials: a first bottom layer made with 138 mg of Avicel PH200, a second separating middle layer made with 456 mg of PEG 1000, a third top layer made with 138 mg of Avicel PH200.

Avicel was used as a model for an active powder that needs to be separated from the fill in a second zone due to mutual incompatibility. PEG is an example of a low melting point material that can be used to form solid separating layers within the present invention.

The fill materials were dispensed as follows: the bottom Avicel layer was dispensed using a volumetric dosing system and compressed thereafter, PEG was then heated to its molten state (above 38 C) and applied evenly onto the bottom Avicel layer using a syringe. Upon contact with the tooling and the powder surface, the PEG cooled, solidified and formed a solid non-compacted layer. Finally, the top Avicel layer was dispensed in the same way as the bottom layer and compressed thereafter to complete the filling step.

Film was applied and thermoformed as described in the method set out in the above Examples.

1. A solid form comprising at least one film enrobing a compacted fill material wherein:
i) said compacted fill material comprises at least one active material;
ii) said solid form shows a weight loss that is less than 1% during a 30 minutes
USP friability test USP 29 Test Number 1216;
iii) said compacted fill material has a density of at least 0.5 g/ml based on the total solid volume of the solid form and a tensile strength of less than 0.9 MPa; and
iv) said compacted fill material comprises at least a first zone and a second zone and said active material is present in at least one of said zones.

2. A solid form comprising at least one film enrobing a compacted fill material wherein said compacted fill material is selected from a pharmaceutical product, a food product, a veterinary product, a cosmetic, an appetite suppressant, a detergent product and a nutraceutical product and comprises at least one active material, the said compacted fill material comprises at least a first zone and a second zone and the said solid form shows a weight loss that is less than 1% during a 30 minutes USP friability test USP 29 Test Number 1216.

3. A solid form according to claim 1 or claim 2 in which the at least one active material has a mean dissolution of at least 75% in 300 seconds in the test specified in the USP Edition 29 Test Number 711 at page 2673 for said active material when the active material is placed in a dissolution medium as specified in the USP dissolution specification or selected from dissolution media specified in the USP according to the solubility properties of the active material or as selected by the skilled person for example selected from: (i) the USP for the at least one active material, (ii) water, (iii) 0.1 M HCl or (iv) phosphate buffer having a pH between 5.8 and 8.0.

4. A solid form according to claim 1 or claim 2 wherein the active material is in a form selected from at least one of granules, micronized powders, spray-dried powders, freeze-dried powders or pellets.

5. A solid form according to claim 1 or claim 2 in which at least one of zone comprises a first active material and a second zone comprises a second active material and wherein the said active materials independently exhibit a release characteristic selected from immediate release, fast release, sustained release, delayed release, controlled release and pulsatile release.

6. A solid form according claim 1 or claim 2 in which at least one of the first zone or second zone of said compacted fill material is in the form of a layer.

7. A solid form according to claim 6 wherein at least one of said layers is a processing aid layer.

8. A solid form according to claim 6 wherein at least one of said layers is selected from an anti-sticking layer, a cushioning layer and a bulking layer.

9. A solid form according to claim 6 wherein at least one of said layers comprises said at least one active material and said layer is an immediate release layer, a fast release layer, a sustained release layer, a controlled release layer, a delayed release layer or a pulsatile release layer.

10. A solid form according to claim 6 wherein at least one of said layers is selected from a separating layer, disintegrant layer, aesthetic layer or a stability enhancing layer.

11. A solid form according to claim 6 wherein at least one of said layers comprises a low dose active material.

12. A solid form according to claim 11 comprising a bulking layer in intimate contact with the low dose active layer.

13. A solid form according to claim 11 wherein said low dose active layer is entrapped between two bulking layers.

14. A solid form according to claim 6, wherein said active material is present in at least two layers.

15. A solid form according to claim 6, wherein said compacted fill material is present in said solid form in at least three layers.

16. A solid form according to claim 6, wherein each layer comprises an active material.

17. A solid form according to claim 1 or claim 2 wherein said at least one active material is a pharmaceutical active.

18. A solid form according to claim 17, wherein said active material comprises at least one of an analgesic, antiangina, antianaemia, antibiotic, antiarrhythmic, antidiarrheal, antidiuretic, antidepressant, antiemetic, antifungal, antirheumatic, antiviral, antiprotozoal, antihistamine, antihypertensive, anti-inflammatory, antimigraine, antinausea, antispasmodic, anxiolytic, beta blocker, calcium channel blocker, sedative, hypnotic, antipsychotic, bronchodilator, decongestant, cough expectorant, cough suppressant, antiasthma drug, corticosteroid, actives for treatment of cough or common cold, muscle relaxant, erectile dysfunction active or motion sickness active.

19. A solid form according to claim 17 comprising at least two active materials wherein the active materials are selected from:
i) an antibiotic in combination with a decongestant, an anti-inflammatory, a cough expectorant, a cough suppressant or an active for treatment of cough or common cold, a proton pump inhibitor;
ii) an anti-HIV, anti-malaria active material, an anti-hypertension and anti-cholesterol,
iii) two or more active materials from the same class of active materials, the class being selected from respiratory actives, gastronintestinal actives, cardiovascular actives, antidiabetes actives, central nervous system actives, anti-infection actives, anti-viral actives, analgesics, anti-inflammatory actives, antibiotics, cough suppressants, expectorants, mucolytics, and nasal decongestants.

20. A solid form according to claim 17 wherein the said at least one active material comprises at least one of paracetamol, pseudoephedrine, acravastine, lamivudine, abacavir, pravastatin, Roziglitazone, ezetimibe, Clavulanate, sulfamethoxazole, benazepril, Valsartan, Irbesartan, Losartan, Dutasteride, tamsolusin, Atazanavir, ritonavir, propoxyphene, Hydrocodone, Metocarbamol, Memantine, Donepezil, Glyburide, Pioglytazone, Glimepiride, Benazepril, Torcetrapib, Eprosartan, Telmisartan, Olmesartan, Lopinavir, Emtricitabine, Tenofovir, Amprenavir, Tipranavir, Atovaquone, Proguanil, 5-aminosalicylic acid, 4-aminophthalic acid, Bismuth citrate, Bismuth subsalicylate, Montelukast, pseudoephedrine, Guaifenesin, ibuprofen, nifedipine, betamethasone acetate, methylprednisolone, dextromethorphan, cinnarazine, simvastatin, ciprofloxacin, glipizide, risperidone, glibenclamide, fenofibrate, isosorbide mononitrate, isosorbide dinitrate, acetazolamide, levothyroxine sodium, omeprazole, aspirin, codeine, dihydroergotamine, diazepam, theophylline, sildenafil citrate, vardenafil hydrochloride, amlodipine besylate, zolpidem tartrate, acetaminophen, methocarbamol, ramipril, digoxin, enalapril maleate, fluoxetine hydrochloride, fexofenadine hydrochloride, olanzapine, methyldopa, hydrochlorothiazide, timolol maleate, alendronate sodium, thiabendazole, rofexocib, dicoflenac, bepridil hydrochloride, atorvastatin hydrochloride, sertraline hydrochloride, famciclovir monohydrate, nabumetone, cimetidine, ketoprofen, etodolac, amiodarone hydrochloride, indomethacin, cefaclor, diltiazem, verapamil, felodipine, isradipine, nicardipine, prazosin, disopyramide, pentoxifilline, venlafaxine, alfuzosin, doxazosin, famotidine, ranitidine, pirenzipine, lansoprazole, loperamide, sulfasalazine, prednisolone, furosemide, amiloride, triamterene, verapamil, atenolol, propranolol, captopril, glyceryl trinitrate, caffeine, aminophylline, cetirizine, loratadine, chlorpheniramine maleate, diphenhydramine, dothiepin, amitriptyline, phenelzine, paroxetine, fenfluramine, dimenhydrinate, ondansetron, domperidone, metoclopramide, tramadol, dihydrocodeine, pethidine, sumatriptan, amoxicillin, ampicillin, cefuroxime, cephalexin, tetracycline, erythromycin, co-trimoxazole, sulphadiazine, trimethoprim, nitrofurantoin, fluconazole, ketoconazole, acyclovir, zidovudine, chloroquine, mefloquin, metronidazole, metformin, chlorpropamide, ferrous sulphate, azapropazone, fenbufen, flurbiprofen, ketoprofen, naproxen, piroxicam, mefanamic acid, celecoxib, licofelone, tadalafil, mycophenolate, valgancyclovir, valacyclovir, sevelamer, metaxolone, nelfinavir, duranavir, tipranavir, levetiracetam, capecitabine, moxifloxacin, morphine, levofloxacin, clarithromycin, pregabalin, esomeprazole, quetiapine, efavirenz, oxcarbazepine, colesevelam, zileuton, nitazoxanide, clofibrate, praziquantel, sucralfate, cefprozil, indinavir, ganciclovir, oxaprozin, divalproex, cefadroxil, felbamate, potassium chloride, saquinavir, fosamprenavir, hydroxyurea, gabapentin, niacin, omega-3 acid ethyl esters, calcium acetate, progesterone, procainamide, delavirdine, ribavirin, propafenone, eprosartan, tocamide, tinidazole, choline magnesium trisalicylate, azithromycin, linezolid, lorazepam, oxazepam, lormetazepam, flunitrazepam, haloperidol, triptorelin, leuprorelin, lanreotide acetate, octreotide acetate, methylxanthin, tamsulosin, codeine hydrochloride, dextromoramide tartrate, ethymorphine hydrochloride, magnesium salicylate, methadone hydrochloride, oxycodone hydrochloride, sufentanil citrate, ephedrine, tramazoline hydrochloride, brompheniramine maleate, emedastine fumarate, and pharmaceuticaly or nutraceuticaly acceptable salts, acids, esters, isomers, and metabolites thereof.

21. A solid form according to claim 17, comprising at least two active materials wherein the active materials are selected from: paracetamol and caffeine; aspirin and paracetamol; paracetamol and pseudoephedrine; paracetamol and phenylephrine; ibuprofen and codeine; ibuprofen and pseudoephedrine; paracetamol and diphenhydramine; acravistine and pseudoephedrine; paracetamol and dextromethorphan; paracetamol and guaphenesin; paracetamol, caffeine, aspirin; aspirin and caffeine; zidovudine, iamivudine and abacavir; pravastatin and aspirin; lamivudine and zidovudine; roziglitazone and metformin; ezetimibe and fenofibrate; amoxicillin and clavulanate; trimetoprim and sulfamethoxazole; amlodipine and benazepril; valsartan and hydrochlorothiazide; irbesartan and hydrochlorothiazide; losartan and hydrochlorothiazide; fenofibrate and metformin; abacavir and lamivudine; dutasteride and tamsolusin; atazanavir and ritonavir; ritonavir and saquinavir; propoxyphene and paracetamol; hydrocodone and paracetamol; tramadol and paracetamol; metocarbamol and paracetamol; memantine and donepezil; glyburide and metformin; pioglytazone and metformin; rosiglitazone and glimepiride, benazepril and hydrochlorothiazide; atorvastatin and torcetrapib; eprosartan and hydrochlorothiazide; amlodipine and atorvastatin; ezetimibe and simvastatin; telmisartan and hydrochlorothiazide; olmesartan and hydrochlorothiazide; lopinavir and ritonavir; emtricitabine and tenofovir; fosamprenavir and ritonavir; amprenavir and ritonavir; tipranavir and ritonavir; atovaquone and proguanil; lansoprazole, amoxicillin and clarithromycin; lansoprazole and naproxen; 5-aminosalicylic acid, 4-aminophthalic acid; clarithromycin, ranitidine and bismuth citrate; bismuth subsalicylate, metronidazole and tetracycline; montelukast and loratadine; fexofenadine and pseudoephedrine; Guaifenesin and pseudoephedrine.

22. A solid form according to claim 1 or claim 2 wherein the dose form contains at least one active material in an amount greater than 100 mg.

23. A solid form according to claim 1 or claim 2 wherein the dose form contains at least one active material in an amount less than 100 mg.

24. A solid form according to claim 1 or claim 2 wherein the tensile strength of the compacted fill material is less than 0.2 MPa.

25. A solid form according to claim 1 or claim 2, wherein the film enrobing the compacted fill material is a water-soluble film.

26. A solid form according to claim 1 or claim 2, comprising a non-compacted fill material in a non-compacted zone interposed between the first and second zone whereby the compacted fill material within the first zone is physically separated from the compacted fill material of the second zone.

27. A solid form according to claim 26, wherein the non-compacted fill material is selected from at least one of polyethylene glycol, polyethylene oxide, polymethacrylates, polyvinyl alcohol, stearic acid, cetyl alcohol, hydrogenated oils, glyceryl behenate, glyceryl palmitostarate, glyceryl monostearate, waxes, or heat stable sugar alcohols.

28. A method of making a solid form according to claim 1 or claim 2 comprising at least one film enrobing a compacted fill material comprising:
i) providing a first film shaped to define an interior volume for holding said compacted fill material and having an open end;
ii) depositing via the open end a first zone of fill material to be compacted in the interior volume and optionally applying pressure to the fill material so as to compact the first zone of fill material;
iii) depositing a second zone of fill material to be compacted in the interior volume such that the interior volume comprises two zones of fill material wherein at least one of the fill material in the first or second zone comprises an active material;
iv) applying pressure to the fill material so as to compact the at least second zone of fill material so forming the said compacted fill material;
v) applying a second film over the said open end to close the said open end; and
vi) sealing the first and second film together to enrobe the compacted fill material and provide the solid form.

29. A method according to claim 28 wherein each zone is in the form of a layer.

30. A method according to claim 28 wherein at least two zones are deposited in a single step.

31. A method according to claim 28 wherein each zone is deposited sequentially and compacted after each deposition.

32. A method according to claim 28 wherein each zone is deposited sequentially and the zones are compacted in a single compaction step.

33. A method according to claim 28 comprising the step of forming a non-compacted zone between the first zone of fill material and the second zone of fill material.

34. A method according to claim 33 comprising the step of applying a non-compacted fill material to the surface of the fill material in the first zone and depositing the second zone of fill material onto the non-compacted fill material whereby the non-compacted fill material provides a physical barrier separating the fill material of the first and second zones.

35. A method according to claim 28, wherein at least one of the layers are deposited using a volumetric or a vacuum system.

36. A solid form according to claim 1 or claim 2 for use in a method of treatment of the human or animal body by therapy.

37. A method of treatment of the human or animal body by administering a solid form according to claim 1 or claim 2 to the human or animal body.
